(19) United States
(12) Patent Application Publication
(10) Pub. No.: US 2026/0069652 A1
(43) Pub. Date: Mar. 12, 2026

(54) LIVER HEALTH SUPPLEMENT FOR COMPANION ANIMALS

(71) Applicant: Jang Wook YEO, Seoul (KR)

(72) Inventor: Jang Wook YEO, Seoul (KR)

(21) Appl. No.: 19/320,377

(22) Filed: Sep. 5, 2025

(30) Foreign Application Priority Data
Sep. 6, 2024 (KR) 10-2024-0121240
Apr. 16, 2025 (KR) 10-2025-0049639

(51) Int. Cl. A61K 38/06; A61K 31/121; A61K 31/355; A61K 31/357; A61K 31/7076; A61K 36/06; A61K 36/28; A61P 1/16

(52) U.S. Cl. CPC A61K 38/063; A61K 31/121; A61K 31/355; A61K 31/357; A61K 31/7076; A61K 36/06; A61K 36/28; A61P 1/16

(57) ABSTRACT

Provided is a liver health supplement for companion animals comprising a S-adenosyl-L-methionine component, a silymarin component, a glutathione component, a vitamin E component, and a curcumin component.

### CROSS-REFERENCE TO RELATED APPLICATION(S)

This application claims priority to Korean Application Nos. 10-2024-0121240, filed Sep. 6, 2024, and 10-2025-0049639, filed Apr. 16, 2025, the disclosures of which are hereby incorporated by reference in their entirety.

### TECHNICAL FIELD

The present invention relates to a liver health supplement for companion animals.

### BACKGROUND ART

Liver diseases in companion animals, comprising conditions like hepatitis, fatty liver disease, and cirrhosis, may lead to serious health problems and even death if left untreated. Conventional treatments often involve medications with potential side effects, and there is a growing demand for natural supplements that can effectively support liver health.

Liver diseases (e.g., chronic liver disease, hepatitis, and liver damage due to oxidative stress) are becoming more prevalent in companion animals. One widely used treatment to support liver health in companion animals is S-adenosyl-L-methionine (SAMe). SAMe is known to improve liver enzyme levels and promote liver cell regeneration. However, there is a need for a more comprehensive liver support supplement that combines multiple components to provide enhanced efficacy.

### SUMMARY OF THE INVENTION

The present invention has been made in an effort to provide a novel liver health supplement for companion animals designed to enhance liver function, reduce oxidative stress, and improve overall liver health. The liver health supplement of the present invention utilizes a combination of natural components proven to be effective in protecting and regenerating the liver. The supplement comprises a liver health supplement for companion animals comprising a S-adenosyl-L-methionine (SAMe) component, a silymarin component, a glutathione component, a vitamin E component, and a curcumin component.

This unique combination improves liver enzyme reduction in companion animals, enhances antioxidant activity, and provides superior benefits for overall liver health compared to SAMe alone. Further, this unique combination improves liver enzyme levels, protects hepatocytes from oxidative damage, and enhances the liver's detoxification process. The combination of SAMe and other hepatoprotectors exhibits a synergistic effect, improving liver function, reducing oxidative stress, and improving liver enzyme levels more effectively than SAMe alone.

The liver health supplement of the present invention may be administered orally in capsule form, and an appropriate dosage is recommended based on the weight of the companion animal.

The combination of components of the liver health supplement of the present invention has a synergistic effect, providing better liver protection and enzyme reduction, as compared to SAMe alone.

The use of water-soluble curcumin as the curcumin component and SAMe-enriched yeast as the SAMe component in the liver health supplement of the present invention increases the absorption rate of the active ingredients, thereby maximizing the effect.

The liver health supplement of the present invention is particularly effective in reducing elevated levels of liver enzymes such as alanine aminotransferase (ALT) and alkaline phosphatase (ALP), which indicate liver damage or dysfunction.

By targeting multiple pathways of liver damage and repair, the liver health supplement of the present invention provides a broader range of benefits than conventional single-component supplements.

### BRIEF DESCRIPTION OF THE DRAWINGS

FIG. 1 is a graph showing the results of a clinical trial using a liver health supplement according to the present invention.

### DETAILED DESCRIPTION

For the purposes of the present specification and claims, unless otherwise indicated, all numbers expressing quantities, percentages or ratios, and other numerical values used in the specification and claims are to be understood as being modified in all instances by the term “about.” The use of the term “about” applies to all numerical values, whether or not explicitly stated. This term generally refers to a range of numbers that a person skilled in the art would consider a reasonable amount of deviation from the stated number (i.e., having an equivalent function or result). For example, this term could be interpreted to encompass a deviation of ±10%, alternatively ±5%, alternatively ±1%, alternatively ±0.5%, and alternatively ±0.1% for a given numerical range, all of which do not alter the ultimate function, effect, or result of that numerical range. Accordingly, unless indicated to the contrary, the numerical parameters set forth in the present specification and claims are approximations that may vary depending upon the desired properties obtained by the present invention.

For the purposes of the present specification and claims, the term “and/or” refers to the listed components, features, etc., alone or in any combination thereof.

### <Components of the Supplement>

The liver health supplement for companion animals according to the present invention comprises a S-adenosyl-L-methionine (hereinafter, “SAMe”) component, a silymarin component, a glutathione component, a vitamin E component, and a curcumin component:
S-adenosyl-L-methionine (hereinafter, “SAMe”) component (e.g., SAMe-enriched yeast) for improving bioavailability and liver support;
Silymarin component (e.g. milk thistle extract) for protecting liver cells from oxidative stress;
Glutathione component (e.g., glutathione-containing yeast) for increasing intracellular antioxidant levels;
Vitamin E component for providing antioxidant effects; and
Curcumin component (e.g., turmeric extract) for high bioavailability and anti-inflammatory effects.

For example, SAMe is provided in-enriched yeast form, which improves its stability and bioavailability in the companion animal's gastrointestinal tract. This allows for more effective liver support, detoxification, and glutathione production.

Silymarin increases the activity of detoxifying enzymes to protect liver cells from oxidative damage and inhibits the ingress of toxic substances into the liver to support overall liver health. Silymarin may be provided, for example, in the form of milk thistle extract.

Vitamin E protects liver cells from oxidative damage and supports overall cellular health to complement the effects of glutathione and silymarin.

Curcumin, which is the active ingredient in turmeric, is provided in a water-soluble form for enhanced bioavailability. (Water-soluble curcumin is absorbed 30 times better than regular curcumin). Curcumin offers potent anti-inflammatory and antioxidant effects, further preventing liver damage and supporting liver regeneration.

### Embodiment

### S-Adenosyl-L-Methionine (SAMe)-Enriched Yeast: 50 to 400 mg

SAMe is a natural metabolite of hepatocytes that supports the methylation process essential for regulating gene expression, neurotransmitter production, and brain function. Yeast-derived SAMe has better bioavailability and absorption than synthetic forms.

Glutathione-Containing Yeast: About 50 to about 500 mg

Glutathione is an important antioxidant that protects liver cells from oxidative stress and supports detoxification. The addition of glutathione to the yeast matrix improves its stability and bioavailability.

Milk Thistle Extract: About 10 to about 220 mg

Silymarin, which is the active ingredient of milk thistle, inhibits the penetration of toxins, increases detoxification enzyme activity, and reduces inflammation to protect liver cells.

Vitamin E: About 2 to about 66 mg

Vitamin E is a powerful antioxidant that protects cell membranes from oxidative damage and supports immune function.

Water-Soluble Curcumin: About 3 mg to about 11 mg

Water-soluble curcumin is the water-soluble form of curcumin, which is the active ingredient of turmeric, and exhibits potent antioxidant and anti-inflammatory properties. Water-soluble curcumin is 30 times more absorbable than regular curcumin.

### <Preparation Method>

The components are mixed in a specified ratio (e.g., parts by weight), encapsulated in gelatin or vegetable capsules, and packaged in blisters to maintain freshness and stability.

### <Clinical Trial>

The components per capsule of the liver health supplement for companion animals formulated for a clinical trial are as follows:

TABLE 1

| Component Name | Content Per Capsule | |
|---|---|---|
| S-adenosyl-L-methionine (SAMe)- | 200 | mg |
| enriched yeast | | |
| Milk thistle extract | 100 | mg |
| Glutathione-containing yeast | 170 | mg |
| Vitamin E | 10 | mg |
| Water-soluble curcumin | 3 | mg |

### <Dosage and Administration>

Companion animals weighing less than 10 kg: One capsule administered orally daily.

Companion animals weighing between 10 and 20 kg: Two capsules administered orally daily.

Companion animals weighing over 20 kg: Three capsules administered orally daily.

The results of administering the liver health supplement for companion animals prepared as above to dogs diagnosed with liver function problems at a domestic pet hospital are as follows:

TABLE 2

| | | | | Dose | ALP | | |
|---|---|---|---|---|---|---|---|
| | | | | Pe- | | | Reduc- |
| | | | Weight | riod | Pre- | Post- | tion |
| Case | Hospital | Age | (kg) | (M) | dose | dose | Rate |
| 1 | Hospital P at | 13 | 4.5 | 1 | 183 | 57 | −69% |
| | Woolsan | | | | | | |
| 2 | Hospital H at | 11 | 7 | 1 | 520 | 162 | −69% |
| | Dongdaemun- | | | | | | |
| | gu | | | | | | |
| 3 | Hospital H at | 5 | 9.8 | 4 | 677 | 231 | −66% |
| | Dongdaemun- | | | | | | |
| | gu | | | | | | |
| 4 | Hospital M at | 11 | 3.8 | 2 | 135 | 79 | −41% |
| | Gangseo-gu | | | | | | |
| 5 | Hospital L at | 3 | 2.6 | 4 | 33 | 15 | −55% |
| | Hwasung | | | | | | |
| 6 | Hospital L at | 12 | 2.8 | 3 | 79 | 77 | −3% |
| | Hwasung | | | | | | |
| 7 | Hospital M at | 16 | 4 | 4 | 264 | 76 | −71% |
| | Cheongju | | | | | | |
| 8 | Hospital J at | 7 | 7.2 | 1 | 89 | 43 | −52% |
| | Jungrang-gu | | | | | | |
| 9 | Hospital Y at | 8 | 6.6 | 1 | 231 | 104 | −55% |
| | Choongju | | | | | | |
| 10 | Hospital D at | 10 | 10 | 1 | 81 | 42 | −48% |
| | Busan | | | | | | |
| 11 | Hospital O at | 7 | 5 | 1 | 384 | 52 | −86% |
| | Busan | | | | | | |
| 12 | Hospital R at | 7 | 17 | 1 | 175 | 121 | −31% |
| | Seongnam | | | | | | |
| 13 | Hospital R at | 12 | 6.7 | 3 | 75 | 71 | −5% |
| | Yongin | | | | | | |
| 14 | Hospital B at | 11 | 4 | 4 | 66 | 43 | −35% |
| | Seongbuk-gu | | | | | | |
| 15 | Hospital J at | 12 | 2.9 | 2 | 270 | 80 | −70% |
| | Woolsan | | | | | | |
| 16 | Hospital A at | 12 | 3.3 | 1 | 205 | 100 | −51% |
| | Woolsan | | | | | | |
| 17 | Hospital C at | 11 | 5 | 2 | 201 | 102 | −49% |
| | Woolsan | | | | | | |
| 18 | Hospital A at | 1 | 3.7 | 1 | 3,109 | 352 | −89% |
| | Cheonan | | | | | | |
| 19 | Hospital A at | 6 | 4.2 | 1 | 1,661 | 305 | −82% |
| | Cheonan | | | | | | |
| | Average | 9.2 | 5.79 | 2 | 444 | 111 | −75% |

TABLE 3

| | | | | | AST | | |
|---|---|---|---|---|---|---|---|
| | | | | Dose | | | Reduc- |
| | | | Weight | Period | Pre- | Post- | tion |
| Case | Hospital | Age | (kg) | (M) | dose | dose | Rate |
| 1 | Hospital H at | 5 | 9.8 | 4 | 54 | 14 | −74% |
| | Dongdaemun- | | | | | | |
| | gu | | | | | | |
| 2 | Hospital M at | 16 | 4 | 4 | 89 | 27 | −69% |
| | Cheongju | | | | | | |
| | Average | 11 | 6.9 | 4.0 | 72 | 21 | −71% |

TABLE 4

| | | | | | ALP | | |
|---|---|---|---|---|---|---|---|
| | | | | Dose | | | Re- |
| | | | | Pe- | | | duc- |
| | | | Weight | riod | Pre- | Post- | tion |
| Case | Hospital | Age | (kg) | (M) | dose | dose | Rate |
| 1 | Hospital P at | 13 | 4.5 | 1 | 325 | 131 | −60% |
| | Woolsan | | | | | | |
| 2 | Hospital P at | 6 | 7.0 | 1 | 280 | 170 | −39% |
| | Daegu | | | | | | |
| 3 | Hospital H at | 11 | 7.0 | 1 | 424 | 240 | −43% |
| | Dongdaemun- | | | | | | |
| | gu | | | | | | |
| 4 | Hospital H at | 2 | 9.8 | 4 | 2,388 | 1,000 | −58% |
| | Dongdaemun- | | | | | | |
| | gu | | | | | | |
| 5 | Hospital M at | 11 | 3.8 | 2 | 1,092 | 540 | −51% |
| | Gangseo-gu | | | | | | |
| 6 | Hospital L at | 12 | 2.8 | 3 | 201 | 158 | −21% |
| | Hwasung | | | | | | |
| 7 | Hospital M at | 16 | 4.0 | 4 | 213 | 111 | −48% |
| | Cheongju | | | | | | |
| 8 | Hospital J at | 7 | 7.2 | 1 | 222 | 106 | −52% |
| | Jungrang-gu | | | | | | |
| 9 | Hospital D at | 10 | 10.0 | 1 | 1,000 | 864 | −14% |
| | Busan | | | | | | |
| 10 | Hospital O at | 7 | 5.0 | 1 | 258 | 92 | −64% |
| | Busan | | | | | | |
| 11 | Hospital R at | 12 | 6.7 | 3 | 849 | 770 | −9% |
| | Yongin | | | | | | |
| 12 | Hospital B at | 11 | 4.0 | 4 | 714 | 529 | −26% |
| | Seongbuk-gu | | | | | | |
| 13 | Hospital J at | 12 | 2.9 | 2 | 300 | 218 | −27% |
| | Woolsan | | | | | | |
| 14 | Hospital P at | 9 | 3.4 | 1 | 313 | 215 | −31% |
| | Woolsan | | | | | | |
| 15 | Hospital A at | 12 | 3.3 | 1 | 401 | 100 | −75% |
| | Woolsan | | | | | | |
| 16 | Hospital C at | 11 | 5.0 | 2 | 385 | 211 | −45% |
| | Woolsan | | | | | | |
| 17 | Hospital A at | 1 | 3.7 | 1 | 102 | 80 | −22% |
| | Cheonan | | | | | | |
| 18 | Hospital A at | 6 | 4.2 | 1 | 549 | 74 | −87% |
| | Cheonan | | | | | | |
| | Average | 9 | 5.2 | 1.9 | 556 | 312 | −44% |

TABLE 5

| | | | | | GGT | | |
|---|---|---|---|---|---|---|---|
| | | | | Dose | | | Reduc- |
| | | | Weight | Period | Pre- | Post- | tion |
| Case | Hospital | Age | (kg) | (M) | dose | dose | Rate |
| 1 | Hospital P at | 6 | 7 | 1 | 30 | — | −100% |
| | Daegu | | | | | | |
| 2 | Hospital H at | 2 | 9.8 | 4 | 336 | 43 | −87% |
| | Dongdaemun- | | | | | | |
| | gu | | | | | | |
| 3 | Hospital M at | 16 | 4 | 4 | 5 | 4 | −20% |
| | Cheongju | | | | | | |
| 4 | Hospital O at | 7 | 5 | 1 | 15 | — | −100% |
| | Busan | | | | | | |
| 5 | Hospital A at | 6 | 4.2 | 1 | 68 | 16 | −76% |
| | Cheonan | | | | | | |
| | Average | 7.4 | 6 | 2.2 | 91 | 13 | −86% |

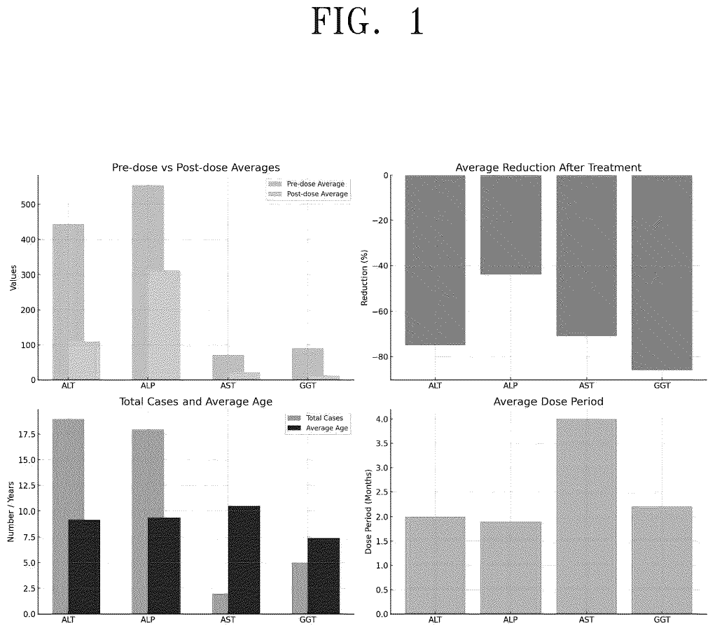

The results of the above-identified clinical trials are summarized in Table 6 and FIG. 1 below.

TABLE 6

| | | | Average | | | |
|---|---|---|---|---|---|---|
| Liver | | | Reduction | | | Av. Dose |
| function | Pre- | Post- | After | Total | Average | Period |
| tests | dose | dose | Treatment | Cases | Age | (M) |
| ALT | 444 | 111 | −75% | 19 | 9.2 | 2.0 |
| ALP | 556 | 312 | −44% | 18 | 9.4 | 1.9 |
| AST | 72 | 21 | −71% | 2 | 10.5 | 4.0 |
| GGT | 91 | 13 | −86% | 5 | 7.4 | 2.2 |

Referring to Table 6 and FIG. 1, it can be confirmed that, in a clinical trial conducted on companion animals suffering from liver disease, the administration of the supplement of the present invention resulted in a significant reduction in major liver enzyme levels (i.e., ALT, AST, ALP, and GGT).

The ALT level was reduced by up to 69% after one month of administration.

The GGT level was reduced by 87% in subjects with elevated liver enzymes.

These results demonstrate that the liver health supplement of the present invention is more effective in improving liver health than SAMe alone.

It's noteworthy that ALT, AST, and GGT were reduced by 75%, 71%, and 76%, respectively, as compared to before being administered with the liver health supplement, over an average of 2 to 4 months, and ALP was reduced only by 44%, which is considered to have the following reasons:
Diverse enzyme functions and origins: ALT is found primarily in the liver and is more directly associated with liver cell damage, making it a more immediate indicator of liver health. In contrast, ALP is found in various tissues, comprising the liver, bones, kidneys, and bile ducts. The ALP level may be influenced by factors unrelated to liver function, such as bone metabolism and bile duct obstruction, making it more difficult to control with liver-targeted supplements.
Different responses to treatment: Some enzymes, such as ALP, may respond more slowly to treatment because their mechanisms of action are different. Supplements may take longer to show effects on ALP or require different dosages or treatment methods, as compared to ALT.
Underlying Conditions: Companion animals with high ALP levels may have underlying conditions not primarily related to the liver, such as bone growth and bile duct problems. These conditions may not respond well to supplements that primarily target liver function.
Supplement Specificity: The formulation of the liver health supplement in this trial may be more effective in targeting pathways related to ALT reduction than those related to ALP. This may be due to the specific components used in the supplement, which may be better suited to address certain liver conditions than others.

### <Statistical Analysis>

A paired t-test on the ALT reduction data shows the following results:
ALT (n=19)
t-statistics: 2.19
p-value: 0.042

This indicates that there is a statistically significant difference (p<0.05) between the ALT levels before and after treatment, suggesting that treatment with the supplement of the present invention had a significant effect on lowering the ALT level.

In addition, the 95% confidence interval for the average difference between the ALT levels before and after treatment is approximately 13.52 and 652.37. This wide interval reflects the variability in the data, but still allows us to be confident that the true average reduction is significantly greater than zero.
AST (n=2)
t-statistic: 4.81
p-value: 0.131
95% Confidence Interval: (−82.92, 183.92)

This suggests that the reduction of AST is not statistically significant (p>0.05) due to the very small sample size (only 2 cases).
ALP (n=18):
t-statistic: 3.27
p-value: 0.004
0.95% Confidence Interval: (87.05, 402.62)

This indicates that the ALP level was significantly reduced after treatment (p<0.01), with a average reduction ranging from 87 to 403 units.
GGT (n=5):
t-statistic: 1.44
p-value: 0.224
0.95% Confidence Interval: (−72.73, 229.13)

The reduction of GGT is not statistically significant (p>0.05), which may be due to the small sample size.

### <Comparative Data>

In the case of SAMe, which is known to be very useful for improving function, the average improvement effect of ALT is known to be 30 to 50% according to data published in academic journals or papers, but the liver health supplement of the present invention has been proven to have a very significant improvement effect of 75% on average, which shows that the efficacy of the liver health supplement of the present invention has been clinically proven.

The average reduction in liver enzymes, particularly ALT, after administering SAMe to companion animals may vary depending on the study, treatment duration, initial liver condition, and species (e.g., dog and cat). However, a general summary based on existing studies and clinical reports is provided below.

### General Findings of the Study:

Dogs: In clinical studies, dogs with elevated ALT levels due to liver disease experienced an average reduction of 25% to 55% in ALT levels after 30 to 90 days of SAMe treatment.
Cats: Studies in cats are more limited, but some reports show a similar range, typically around 30% to 50% reduction after several weeks of SAMe treatment.

### Summary of Average Reduction:

Overall average reduction: Several studies have shown that the typical average reduction in ALT levels after SAMe use in both dogs and cats is approximately 30% to 50%.

It is confirmed that the administration of the liver health supplement of the present invention showed a significantly greater degree of reduction of ALT than the administration of SAMe alone.

What is claimed is:

1. A liver health supplement for companion animals comprising a S-adenosyl-L-methionine (SAMe) component, a silymarin component, a glutathione component, a vitamin E, component, and a curcumin component.

2. The liver health supplement for companion animals of claim 1, wherein the liver health supplement comprises:
the S-adenosyl-L-methionine (SAMe) component in range of about 40 to to about 400 parts by weight,
the silymarin component in a range of about 10 to about 220 parts by weight,
the glutathione component in a range of about 50 to about 500 parts by weight,
the vitamin E component in a range of about 2 to about 66 parts by weight, and
the curcumin component in a range of about 3 to about 11 parts by weight.

3. A capsule comprising the liver health supplement of claim 1, wherein the capsule comprises:
about 50 to about 400 mg of a S-adenosyl-L-methionine (SAMe)-enriched yeast as the S-adenosyl-L-methionine (SAMe) component;
about 10 to about 220 mg of a milk thistle extract as the silymarin component;
about 50 to about 500 mg of a glutathione-containing yeast as the glutathione component;
about 2 to about 66 mg of vitamin E as the vitamin E component; and
about 3 to about 11 mg of water-soluble curcumin as the curcumin component.

4. The capsule of claim 3, wherein the capsule comprises:
about 200 mg of a S-adenosyl-L-methionine (SAMe)-enriched yeast;
about 100 mg of a milk thistle extract;
about 170 mg of a glutathione-containing yeast;
about 10 mg of vitamin E; and
about 3 mg of water-soluble curcumin.
